(19) United States
(12) Patent Application Publication
(10) Pub. No.: US 2019/0142305 A1
(43) Pub. Date: May 16, 2019

(54) Method for Using Location Tracking Dementia Patients

(71) Applicant: AiCare Corporation, San Jose, CA (US)

(72) Inventors: Sean Tan, San Jose, CA (US); Hsien-Chung Woo, Los Altos Hills, CA (US); John Fee, Garland, TX (US); Helen Balabine, Menlo Park, CA (US)

(21) Appl. No.: 16/180,098

(22) Filed: Nov. 5, 2018

Related U.S. Application Data
(60) Provisional application No. 62/585,243, filed on Nov. 13, 2017.

(51) Int. Cl. A61B 5/11

(52) U.S. Cl. CPC A61B 5/1118; A61B 5/4088; A61B 5/112; A61B 5/1112

(57) ABSTRACT

The present invention includes systems and methods for preventing negative interaction between patients with dementia in a pre-determined area comprising: positioning a plurality of wireless communication devices at specific, known locations in the pre-determined area; providing each patient with dementia with a wearable medical device; providing a patient database; and using a processor in communication with the patient database and the plurality of wireless communication devices, wherein the processor calculates a position in the pre-determined area for each patient, and wherein the processor displays an alert when a movement of two patients that are in the patient database having known negative interaction are approaching each other within a line-of-sight or within a pre-determined distance.

### CROSS-REFERENCE TO RELATED APPLICATIONS

This application claims priority to U.S. Provisional Application Ser. No. 62/585,243, filed Nov. 13, 2017, the entire contents of which are incorporated herein by reference.

### STATEMENT OF FEDERALLY FUNDED RESEARCH

None.

### TECHNICAL FIELD OF THE INVENTION

The present invention relates in general to the field of tracking dementia patients, and more particularly, to a wearable device that tracks the location, gait, and other characteristics of dementia patients.

### BACKGROUND OF THE INVENTION

Without limiting the scope of the invention, its background is described in connection with.

One such device is taught U.S. Patent Publication No. 20160139273, filed by Sobol, and entitled “Wireless Devices and Systems for Tracking Patients and Methods for Using the Like”, which is said to teach apparatuses, systems, and methods for tracking patients that suffer from dementia, that include a a wearable device capable of micro-tracking through Bluetooth Low Energy technology and capable of macro-tracking through GPS technology. The device may additionally include sensors to monitor other information such as the health of the patient or the patient's surrounding environment. These systems are said to also teach how the device interacts with the other components of the system (e.g., signal beacons, wireless transmitters, a central processing unit, mobile computing devices) to provide an integrated system to tracking the location and monitoring the well being of the patient. Finally, methods for tracking patients that use the disclosed devices and systems are said to be disclosed.

Another such device is taught in U.S. Patent Publication No. 20160278652, filed by Kaib, et al., entitled “Systems and Methods of Determining Location Using a Medical Device”. These applicants are said to teach a wearable medical device that may include a sensing electrode to sense an electrocardiogram signal of a patient, a therapy electrode to provide treatment to the patient, a garment to be worn about a torso of the patient and receive the sensing electrode and the therapy electrode, and a controller operatively coupled to the sensing electrode and the therapy electrode. The controller is said to be configured to determine a current location of the wearable medical device based on a previous position of the medical device and at least a speed and a direction of movement of the wearable medical device.

However, despite these devices and methods a need remains for a comprehensive system, apparatus, and methods for tracking and predicting behavior of dementia patients in residential facilities that are cost effective and require minimal or no batteries or other devices that require continuous replacement of batteries or recharging.

### SUMMARY OF THE INVENTION

In one embodiment, the present invention includes a method for preventing negative interaction between patients with dementia in a pre-determined area comprising: positioning a plurality of wireless communication devices at specific, known locations in the pre-determined area; providing each patient with dementia with a wearable medical device equipped to communicate with the plurality of wireless communication devices actively or passively; providing a patient database that comprises specific information for all patients with the wearable medical device, the database further comprising specific information about which patients have dementia, wherein the database includes which patients are likely to have a negative interaction with a dementia patient, and a percent probability that the dementia patient will have a negative interaction with another patient; using a processor in communication with the patient database and the plurality of wireless communication devices, wherein the processor calculates a position in the pre-determined area for each patient, and wherein the processor displays an alert when a movement of two patients that are in the patient database having known negative interaction are approaching each other within a line-of-sight or within a pre-determined distance based on the percent probability. In one aspect, the wearable medical device comprises one or more magnets that are detected by a plurality of magnetic impedance sensors at specific, known locations in the pre-determined area, including triangulation of the position of the patient of patients. In another aspect, the input/output device is a wireless communication is selected from at least one of: IEEE 802.11 (WiFi), IEEE 802.15.4, BLUETOOTH protocol, Near Field Communication (NFC), Radio Frequency Identification (RFID), SIGFOX protocol, WiMax (world interoperability for microwave access), Universal Mobile Telecommunications System (UMTS), 3GPP Long Term Evolution (LTE), IMS, High Speed Packet Access (HSPA), Global System for Mobile communication (GSM), 3G, 4G, 5G, 6G and higher, AM, or FM. In another aspect, the processor connects to a network selected from Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing. In another aspect, the wearable medical device is powered and further comprises at least one of a power source, a display, an input/output device, or a memory. In another aspect, the processor displays or transfers proximity information to staff in pre-determined area via Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing. In another aspect, the processor, the wearable device, or both, further comprise a display and a memory. In another aspect, the position of the patient or patients is augmented with at least one of: a barometer, a pressure sensor, a vibration sensor, an optical sensor, an infrared sensor, a motion sensor, a magnetometer, or a magnetic sensor. In another aspect, the processor is connected to the database via wired or wireless communications, and wherein the database is in the cloud. In another aspect, the wearable device transmit signals to an internet-of-things (IoT) device for data transmission, transmits a beacon, a broadcast signal, or a packet to the processor for determining the location of the patient. In another aspect, the wearable device is worn on a limb or other part of the patient, an accessory worn by the patient, or on a garment worn by the patient. In another aspect, the method further comprises providing a code segment that anticipates, based on a direction of travel of one or more of the patients with dementia in the pre-determined area, when two patients are likely to come in contact, and alerting staff of the possible contact. In another aspect, the method further comprises subdividing the pre-determined area into different genders, levels of dementia, types of dementia, a level of violence associated with a subset of patients. In another aspect, the method further comprises providing a code segment that uses trend analysis to predict at least one of: (1) a long term motion and habit behavior of the one or more patients; (2) trend of a person in the close proximity to another person; (3) determine and track the travel path and location of a specific person; (4) predict where the tracked person may be travelling to a location; or (5) combining trend analysis results to predict a long term motion and habit behavior. In another aspect, the code segment for (1) and (5) uses a linear algebraic analysis, wherein the code segment for (3) uses a graph analysis, or wherein the code segment for (2) and (3) uses a Monte-Carlo-based probabilistic prediction. In another aspect, the method further comprises storing in the database the frequency and extend of an altercation two or more patients. In another aspect, the method further comprises providing a visitor, staff, or family member with a wearable device to track the position of the visitor, staff, or family member.

In another embodiment, the present invention includes a system for preventing negative interaction between patients with dementia in a pre-determined area comprising: a plurality of wireless communication devices at specific, known locations in the pre-determined area; a wearable medical device on each patient with dementia equipped to communicate with the plurality of wireless communication devices actively or passively; a patient database that comprises specific information for all patients with a wearable medical device, the database further comprising specific information about which patients have dementia, wherein the database includes which patients are likely to have a negative interaction with a dementia patient, and a percent probability that the dementia patient will have a negative interaction with another patient; and a processor in communication with the patient database and the plurality of wireless communication devices, wherein the processor calculates a position in the pre-determined area for each patient, and wherein the processor displays an alert when a movement of two patients that are in the patient database having known negative interaction are approaching each other within a line-of-sight or within a pre-determined distance based on the probability. In one aspect, the wearable medical device comprises one or more magnets that are detected by a plurality of magnetic impedance sensors at specific, known locations in the pre-determined area, including triangulation of the position of the patient. In another aspect, the wireless communication is selected from at least one of: IEEE 802.11 (WiFi), IEEE 802.15.4, BLUETOOTH protocol, Near Field Communication (NFC), Radio Frequency Identification (RFID), SIGFOX protocol, WiMax (world interoperability for microwave access), Universal Mobile Telecommunications System (UMTS), 3GPP Long Term Evolution (LTE), IMS, High Speed Packet Access (HSPA), Global System for Mobile communication (GSM), 3G, 4G, 5G, 6G and higher, AM, or FM. In another aspect, the wearable medical device is powered or passive. In another aspect, the processor displays or transfers proximity information to staff in pre-determined area via Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing. In another aspect, the wearable medical device is powered and further comprises at least one of a power source, a display, an input/output device, or a memory. In another aspect, the position of the patient or patients is augmented with at least one of: a barometer, a pressure sensor, a vibration sensor, an optical sensor, an infrared sensor, a motion sensor, a magnetometer, or a magnetic sensor. In another aspect, the processor is connected to the database via wired or wireless communications, and wherein the database is in the cloud. In another aspect, the wearable device transmit signals to the processor for data transmission, transmits a beacon, a broadcast signal, or a packet to the processor for determining the location of the patient. In another aspect, the wearable device is worn on a limb or other part of the patient, an accessory worn by the patient, or on a garment worn by the patient. In another aspect, the system further comprises a code segment that anticipates, based on a direction of travel of one or more of the patients with dementia in the pre-determined area, when two patients are likely to come in contact, and alerting staff of the possible contact. In another aspect, the system further comprises a code segment that uses trend analysis to predict at least one of: (1) a long term motion and habit behavior of the one or more patients; (2) trend of a person in the close proximity to another person; (3) determine and track the travel path and location of a specific person; (4) predict where the tracked person may be travelling to a location; or (5) combining trend analysis results to predict a long term motion and habit behavior. In another aspect, the code segment for (1) and (5) uses a linear algebraic analysis, wherein the code segment for (3) uses a graph analysis, or wherein the code segment for (2) and (3) uses a Monte-Carlo-based probabilistic prediction. In another aspect, the system further comprises a database that stores the frequency and extend of an altercation two or more patients. In another aspect, a visitor, staff, or family member wearable device to track the position of the visitor, staff, or family member.

In another embodiment, the present invention includes a wearable device for preventing negative interaction between patients with dementia in a pre-determined area comprising: a wearable medical device equipped to communicate with the plurality of wireless communication devices actively or passively, wherein the wearable medical device comprises a processor comprising a non-transitory computer readable medium comprising instructions stored thereon for; communicating with a patient database that comprises specific information for all patients with the wearable medical device, the database further comprising specific information about which patients have dementia, wherein the database includes which patients are likely to have a negative interaction with a dementia patient, and a percent probability that the dementia patient will have a negative interaction with another patient; using a processor in communication with the patient database and the plurality of wireless communication devices, wherein the processor calculates a position in the pre-determined area for each patient, and wherein the processor displays an alert when a movement of two patients that are in the patient database having known negative interaction are approaching each other within a line-of-sight or within a pre-determined distance based on the percent probability.

### BRIEF DESCRIPTION OF THE DRAWINGS

For a more complete understanding of the features and advantages of the present invention, reference is now made to the detailed description of the invention along with the accompanying figures and in which:

FIG. 1 shows the basic architecture and design of the system of the present invention.

FIG. 2 is a screenshot of a patient management system of the present invention that includes the patient status, emergency call information, and a map showing patient location.

FIG. 3 shows the detection of two persons that are getting close to each other, which triggers an alert that is generated and sent to web service, app or other communication device to alert the care staff to separate them apart to safe distances.

FIG. 4 shows the location tracking and data collected of the person to obtained the mentioned information for trending and prediction purpose.

FIG. 5A shows the details of a signal distance location and magnetic disturbance location detection system, in which the location of the person can either be detected using the signal strength of the wireless signal in either single IoT or multiple IoT box.

FIG. 5B shows the details of a signal distance location and magnetic disturbance location detection system, in which the location of the person can either be detected using the signal strength of the wireless signal in either single IoT or multiple IoT box that further includes motion and/or vibration sensors.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

For a person that has dementia, it is easy for that person to recognize another person with dementia. With this ability to recognize the other person with or without a mental condition, dementia patients are known to create disturbance and irritation for the other dementia person, which leads to altercations and fights on a regular basis. This is especially problematic for centers with dementia patients or senior centers with dementia seniors.

The present invention includes devices and methods for identifying the location of the dementia person and to alert the caretakers or families is devised to reduce the chance of altercation and to protect the dementia person. The present invention detects the location of each dementia patient or senior and separates them to prevent problems.

As used herein, the term “negative interaction” refers to any trigger that leads to a negative effect on a patient, e.g., a dementia patient and includes a negative interaction between a dementia patient and another dementia patient, the dementia patient and another patient (with or without dementia), or even between the dementia patient a location, a situation, or that triggers a memory or other adverse effect in a mental facility, a healthcare facility, a rehabilitation center (e.g., a drug or other mental health rehabilitation facility), a patient care facility, or other pre-determined location in which patients are able to interact. For example, the negative interaction for a dementia patient may be that the patient has an adverse effect when they see another patient (with or without dementia) that triggers violence, depression, a change in mood or condition (e.g., shock, elevated heart rate, loss of cognition, etc.), that leads to an assault or other adverse effect (e.g., entering an area restricted by age, gender, patient population, staff areas, etc.) on the dementia patient, the other patient, visitors, or staff. The present invention includes an adaptive zone that can change based on additional interactions and locations, e.g., when two patients are known to trigger an adverse effect on a consistent basis, then the adaptive zone can be enlarged to permit staff to prevent the negative interaction. The zone can also be adapted in real-time based on the distance between staff and the dementia patient, the other patient, visitors, or staff (which location may also be monitored or based on a fixed base, e.g., nurse's station). The adaptive zone can also be changed depending on the time of day, e.g., to prevent male or female patients from entering non-designated areas during certain times of the day.

As used herein, the term “dementia patient” refers to any mental health condition that prevents or decreases cognitive reasoning or understanding. The dementia may be the result of genetics, trauma, age, drug use, or other environmental or health conditions that lead to lower cognitive abilities. For example, the patient may have Down's syndrome, Cerebral palsy, age-onset dementia, drug-induced dementia (permanent or temporary), or may have other chronic or acute mental limitations that result in impaired reasoning, and thus, the potential for a negative interaction with a person, circumstances (e.g., loud noise, bright lights, confinement, claustrophobia, fear of animals or insect, touch, being watched, etc.), or a location that triggers a loss of reasoning.

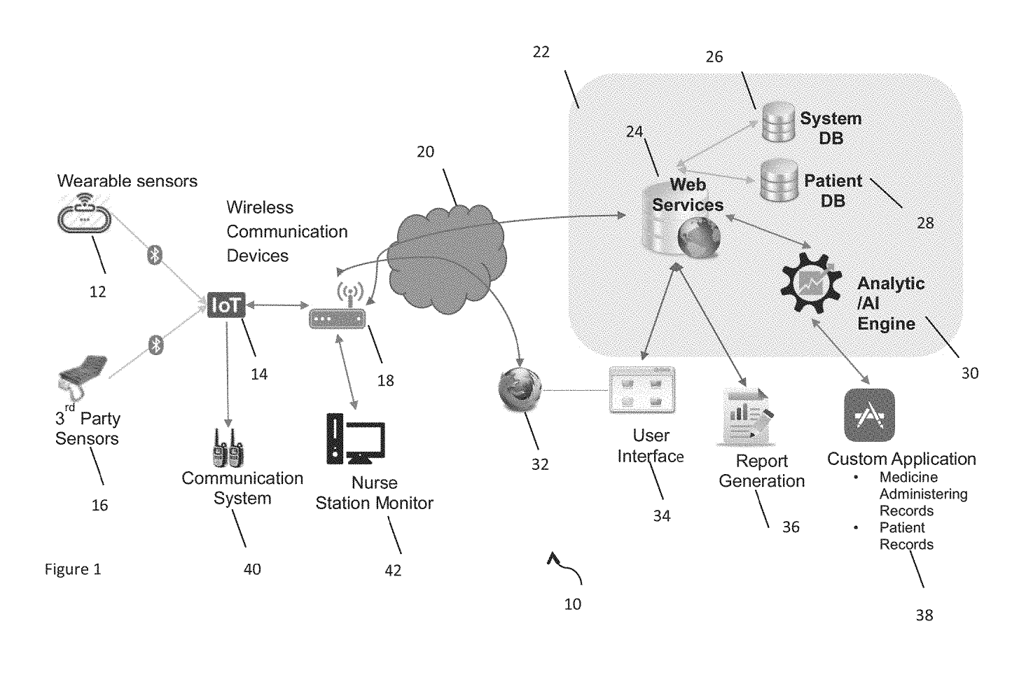

FIG. 1 shows the basic architecture and design of one example of a system 10 for identifying and tracking dementia patients present invention. In FIG. 1, a physical wearable device 12 is placed in communication with an IoT box 14, and one or more sensors 16, for determining the wearable device signal, determining; the magnetic interference, vibration, or other sensors to translate data from the physical wearable device 12 and the sensors 16, via a modem 18 or other communications or switching device to a cloud-based system 20. The cloud based system 22 communicates with a processor 22 that can communicate to web services engine 24, a system database 26, a patient database 28, and an analytical/artificial intelligence engine 30. The web services engine 24 can communicate via a browser 32 to a user interface 34, or the web services system 24 can also communicate directly with the user interface 34. The web services engine 24 can also generate reports. The analytical/artificial intelligence engine 30 can communicate with an application, such as a custom application 38. The processor 22 can also communicate via the cloud 20 through a modem 18 (or hardwired) with a nurses station 42 (which could also be accessed via browser 32 and user interface 34. The modem 18 can also communicate and send alerts to a communications system 40, nurse or healthcare provider station 42, the user interface 34 (e.g., on a mobile device, tablet, or mobile computer), and the report generator 36, all of which can be connected to the cloud 20.

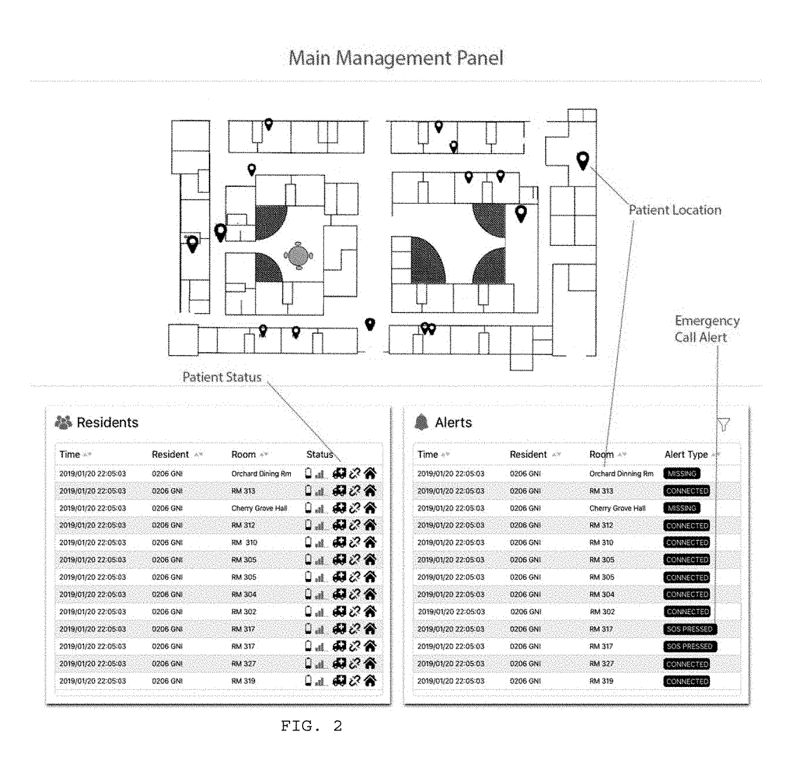

FIG. 2 is a screenshot of a patient management system of the present invention that includes the patient status, emergency call information, and a map showing patient location.

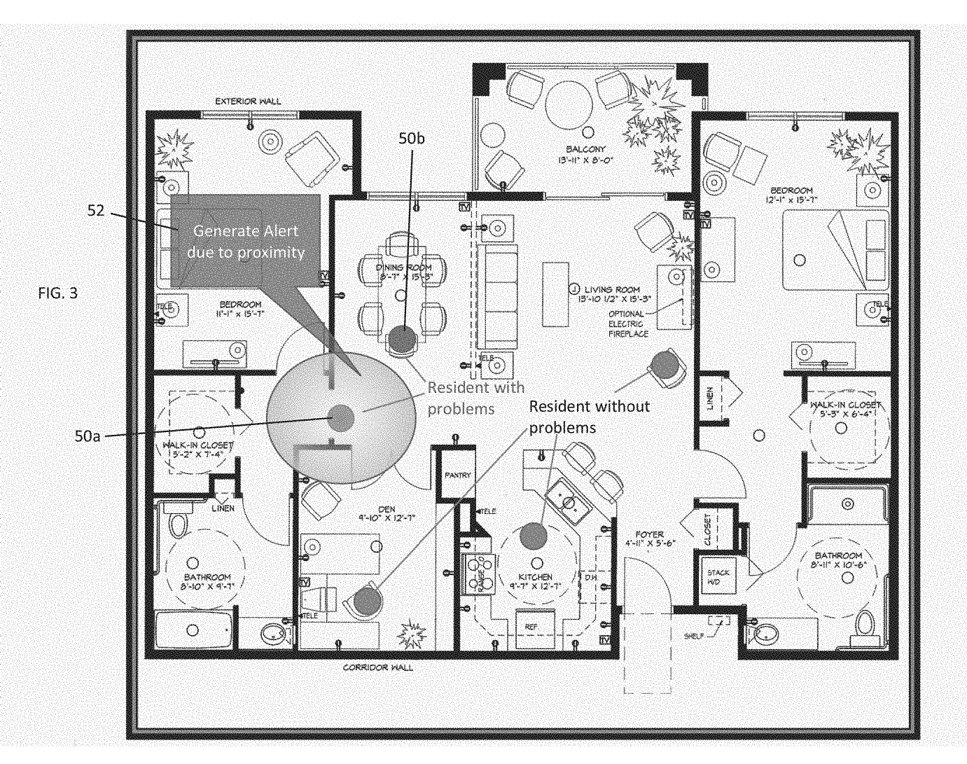

FIG. 3 shows the detection of two tagged persons (50a, 50b) that are getting close to each other. In this embodiment, an alert 52 is generated and send to web service, app or other communication device to alert the care staff to separate them apart to safe distances. Using the system 10, the healthcare provider can be alerted ahead of time (when the two tagged persons (50a, 50b) are approaching each other), to prevent the altercation.

Using the system of the present invention, the normal location(s) and movement(s) of a tracked person is tracked to provide a trend of usual place of visit. This data is tracked 24/7 continuously to provide: (1) healthcare staff awareness of the general habit of the person, (2) a prediction of where the person might be going in the future, (3) tracking the person to not to enter out of bound area, and/or providing evidence of existence of location for future proof or prediction.

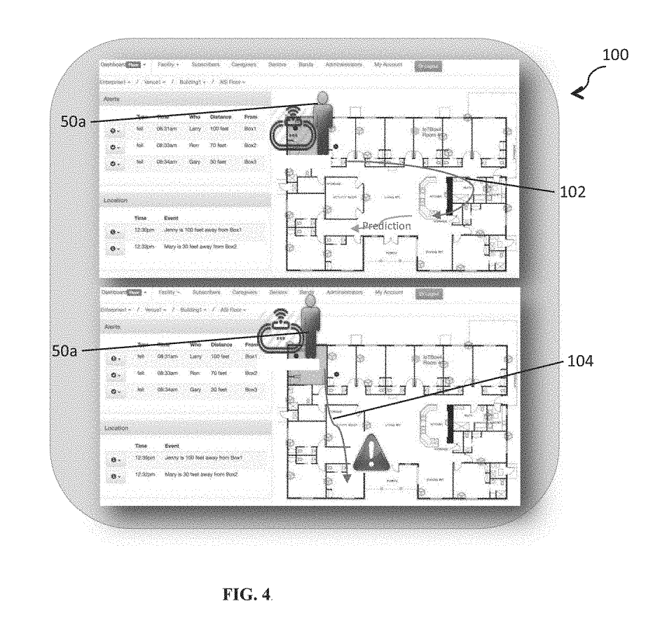

FIG. 4 illustrates the location tracking and data collected of the person to obtained the mentioned information for trending and prediction purpose. In FIG. 4, a layout of a facility 100 in which the tagged person 50a is shown. The top portion shows that the tagged person is at a first position in the facility 100 and based on the patient database (e.g., the tagged person's past history of movement), will follow an expected path 102, which is either typical for the tagged person 50a and/or through which the tagged person 50a will not encounter another tagged person with which an altercation can be a predicted or expected path 102. By contrast, in the bottom portion, the tagged person 50a initiates a path that is a not expected path 104 and/or through which the tagged person 50a will encounter another tagged person with which the tagged person 50a has a prior history of conflict or altercations. The tracking of the tagged person 50a is being done with IoT box placed at specific location. The IoT box can either sense the signal strength of the physical wearable device or from the magnetic disturbance from the physical wearable device or a hybrid of the two. The IoT box can also be connected to magnetic sensors, magnetometers, motion sensors, vibration sensors, pressure sensors, lasers, and/or, infrared sensors, etc., position in high traffic areas, in specific regions, or throughout the facility 100.

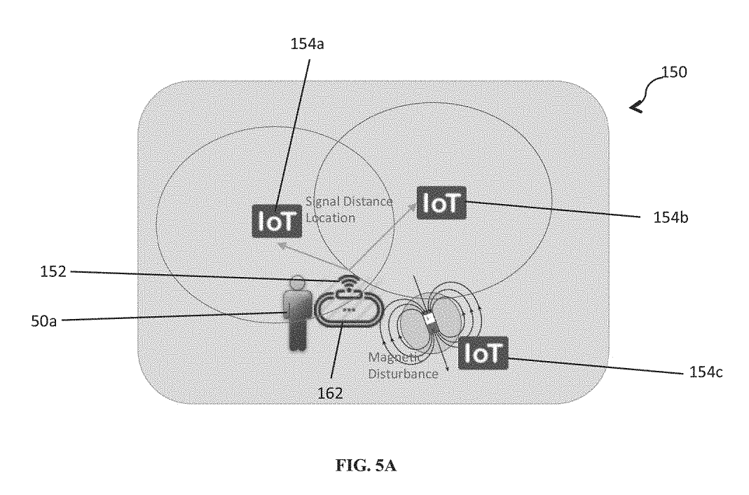

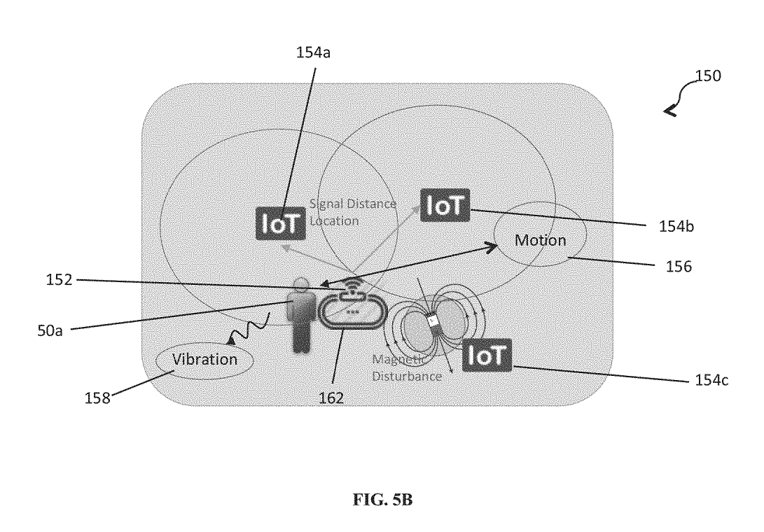

FIG. 5A shows the details of a signal distance location and magnetic disturbance location detection system 150, in which the location of the person can either be detected using the signal strength of the wireless signal 152 in either single IoT or multiple IoT boxes 154a,b,c. In FIG. 5B the details of a signal distance location and magnetic disturbance location detection system 150 is shown, in which the location of the person can either be detected using the signal strength of the wireless signal 152 in either single IoT or multiple IoT boxes 154a,b,c that further includes motion 156 and/or vibration sensors 158 that can also be installed in high traffic areas, in specific regions, or throughout the facility 100 to track and predict the movements of patients within the facility 100. The percent probability of a negative interaction between two patients (one or more of which may be a dementia patient) is calculated into a distance, zone or cone of probability of a negative interaction. This zone or distance may be calculated (and recalculated) for each patient proportional to his or her risk at one or more times. The risk can be learned by the frequency and duration that a patient is outside their zone and dwell in other zones. The probability zone can begin with an initial assessment by the hospital staff and is then built based on daily tracking of each patient (could be an existing dementia patient or a predictive algorithm which looks at all patients and anyone of those who began to show abnormalities our flagged and put on a watchlist). Further changes to the distance or zone are possible of this particular patient and the tracking and monitoring and zoning of that patient is been built to assess the potential risk of possible new dementia patients.

The percent probability can also be used to define a probablistic risk zone around each of the patients (dementia and/or not) as they travel in the building. This risk assessment profile is initially generated for each patient. As the patient(s) move(s) around some of them will exhibit lower risk than others, while and others would show higher risk than the original profile shows.

These risks zones are continuously measured and readjusted for those particular patients who are in this risk profile category. The continuous measurement and readjustment becomes a self-learning system that uses artificial intelligence rules and characteristics to create a more accurate risk profile then the initial assessment. The risk assessment profile is continually updated and honed in so that it becomes more accurate over time.

One or more additional risk profile(s) may include additional measurements that, when the patients approach each other, as the high-risk patients require further distance apart than normal lower risk patients and the alarms are generated much quicker for those ones which are higher risk. The convolution of several of them can create different risk zone probabilities and these profiles all of which are generated via the artificial intelligence self learning algorithm.

FIGS. 5A and 5B demonstrate that the location of the tagged person 50a can either be detected using the signal strength of the wireless signal 152 in either single IoT or multiple IoT boxes 154. The IoT box 154 can also be used to sense the magnetic disturbance 160 created by the physical wearable device 162 to determine the location of the tagged person 50a or a hybrid of the two or three detector system to improve the accuracy.

Thus, in one embodiment, the present invention includes devices and methods for dementia person location tracking and alert is comprising one or more of the following: (1) a physical wearable device equipped with wireless (Bluetooth, WiFi, LTE, GSM and other wireless technology) communication function, (2) a physical wearable device equipped with magnetic sensors, magnetometers, motion sensors, vibration sensors, pressure sensors, lasers, and/or, infrared sensors, etc., as augmentation to the location determination, (3) a physical wearable device equipped with magnetometer for augmentation to the location determination, (4) a physical wearable device equipped with magnetic sensor for augmentation to the location determination, (5) a physical wearable containing magnet for augmentation to the location determination, (6) a physical wearable containing magnetic signature with arrangement with magnetic or other ferrous material, (7) a physical wearable generating modulated magnetic signature for identification, (8) a physical gateway (IoT) device with wireless communication function for communicating with the wearable device and communication function to communicate over wired or wireless communication with the internet for placing in the building, and/or (9) a physical gateway (IoT) device with magnetic sensor for detecting the magnetic field disturbance from the preset magnetic signature and pattern on the physical wearable.

The present invention can also includes one or more of the following: (1) a cloud based system that contains the database of all person wearing the physical wearable device, (2) a cloud based system that communicates with the IoT device tracking the location of each individual person, (3) a physical wearable device that transmit signals to IoT device for data transmission, (4) a physical wearable device transmit beacon, broadcast signal or packet to the IoT device for location determination, (5) an IoT device that receives the signal from physical wearable device to determine the signal strength of the physical wearable device, (6) an IoT device that sends the signal condition information to the cloud based system for analysis, and/or (7) an IoT device that is positioned within the locations where the tracking of the person is desired.

The present invention can also includes one or more of the following: (1) physical wearable device is worn on a person's limb to be used for location determination, (2) a physical wearable device can also be worn on any parts of the body for location determination, (3) a physical wearable device can also be in the form of jewelry, tag, key chain or as an object that can be carried by the person to be located, and/or (4) a physical wearable device can also be part of integrated or placed into clothing of person to be located and tracked.

The present invention can also includes one or more of the following: (1) a cloud based system that correlates data from the physical wearable and IoT device to perform location detection of person using the signal strength difference for closest physical wearable to the IoT device. The cloud based system can be used to further identify or pinpoint (actively or passively): (1) the location of the person by correlating multiple IoT signal information of that particular physical wearable device, (2) the location of the person wearing the physical device by using the barometric information detected by the physical device, (3) the location of the person wearing the physical device with magnetic field signature of the location, (4) the location of the person wearing the physical device with magnetic field disturbance of the location created by pre-set magnetic disturbance signature, and/or (5) the location of the person by using the magnetic sensor to determine the magnetic signature sent from the physical wearable device.

The present invention can also includes one or more of the following: (1) identifying the number of person and who tracked person has come into contact with, (2) labeling the other person that the tracked person has had prior problems or altercations. The system can further provide alerts to the care staff or family member or any other caring person when the tracked person is coming within a predetermined distance of the other person that may cause further problems.

The present invention can also includes one or more of the following: (1) providing alerts when the person enters or leaves pre-determined area, (2) providing alerts when the person enters or leaves opposite sex area, (3) logging all the events in raw data format and stored in the cloud system, (4) providing location evidence of where about the person is or was at a particular time, (5) applying trending analysis to determine the trend of a person in the close proximity to another person (self learning), (6) applying trending analysis to determine and track the travel path and location of a specific person, (7) applying trending analysis to predict where the tracked person may be travelling to a location, and/or (8) combining trend analysis results to predict a long term motion and habit behavior.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for preventing negative interaction between patients with dementia in a pre-determined area comprising:
positioning a plurality of wireless communication devices at specific, known locations in the pre-determined area;
providing each patient with a wearable medical device equipped to communicate with the plurality of wireless communication devices actively or passively;
providing a patient database that comprises specific information for all patients with the wearable medical device, the database further comprising specific information about which patients have dementia, wherein the database includes which patients are likely to have a negative interaction with a dementia patient, and a percent probability that the dementia patient will have a negative interaction with another patient;
using a processor in communication with the patient database and the plurality of wireless communication devices, wherein the processor calculates a position in the pre-determined area for each patient, and
wherein the processor displays an alert when a movement of two patients that are in the patient database having known negative interaction are approaching each other within a line-of-sight or within a pre-determined distance based on the percent probability.

2. The method of claim 1, wherein the wearable medical device comprises one or more magnets that are detected by a plurality of magnetic impedance sensors at specific, known locations in the pre-determined area.

3. The method of claim 1, wherein the input/output device is a wireless communication is selected from at least one of: IEEE 802.11 (WiFi), IEEE 802.15.4, BLUETOOTH protocol, Near Field Communication (NFC), Radio Frequency Identification (RFID), SIGFOX protocol, WiMax (world interoperability for microwave access), Universal Mobile Telecommunications System (UMTS), 3GPP Long Term Evolution (LTE), IMS, High Speed Packet Access (HSPA), Global System for Mobile communication (GSM), 3G, 4G, 5G, 6G and higher, AM, or FM.

4. The method of claim 1, wherein the processor connects to a network selected from Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing.

5. The method of claim 1, wherein the wearable medical device is powered and further comprises at least one of a power source, a display, an input/output device, or a memory.

6. The method of claim 1, wherein the processor displays or transfers proximity information to staff in pre-determined area via Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing.

7. The method of claim 1, wherein the processor, the wearable device, or both, further comprise a display and a memory.

8. The method of claim 1, wherein the position of the patient or patients is augmented with at least one of: a barometer, a pressure sensor, a vibration sensor, an optical sensor, an infrared sensor, a motion sensor, a magnetometer, or a magnetic sensor.

9. The method of claim 1, wherein the processor is connected to the database via wired or wireless communications, and wherein the database is in the cloud.

10. The method of claim 1, wherein the wearable device transmit signals to IoT device for data transmission, transmits a beacon, a broadcast signal, or a packet to the processor for determining the location of the patient.

11. The method of claim 1, wherein the wearable device is worn on a limb or other part of the patient, an accessory worn by the patient, or on a garment worn by the patient.

12. The method of claim 1, further comprising providing a code segment that anticipates, based on a direction of travel of one or more of the patients with dementia in the pre-determined area, when two patients are likely to come in contact, and alerting staff of the possible contact.

13. The method of claim 1, further comprising subdividing the pre-determined area into different genders, levels of dementia, types of dementia, a level of violence associated with a subset of patients leading to verbal, mental, or physical violence or bullying.

14. The method of claim 1, further comprising providing a code segment that uses trend analysis to predict at least one of: (1) a long term motion and habit behavior of the one or more patients; (2) trend of a person in the close proximity to another person; (3) determine and track the travel path and location of a specific person; (4) predict where the tracked person may be travelling to a location; or (5) combining trend analysis results to predict a long term motion and habit behavior.

15. The method of claim 14, the code segment for (1) and (5) uses a linear algebraic analysis, wherein the code segment for (3) uses a graph analysis, or wherein the code segment for (2) and (3) uses a Monte-Carlo-based probabilistic prediction.

16. The method of claim 1, further comprising storing in the database the frequency and extend of an altercation two or more patients.

17. The method of claim 1, further comprising providing a visitor, staff, or family member with a wearable device to track the position of the visitor, staff, or family member.

18. A system for preventing negative interaction between patients with dementia in a pre-determined area comprising:
a plurality of wireless communication devices at specific, known locations in the pre-determined area;
a wearable medical device on each patient equipped to communicate with the plurality of wireless communication devices actively or passively;
a patient database that comprises specific information for all patients with a wearable medical device, the database further comprising specific information about which patients have dementia, wherein the database includes which patients are likely to have a negative interaction with a dementia patient, and a percent probability that the dementia patient will have a negative interaction with another patient; and
a processor in communication with the patient database and the plurality of wireless communication devices, wherein the processor calculates a position in the pre-determined area for each patient, and wherein the processor displays an alert when a movement of two patients that are in the patient database having known negative interaction are approaching each other within a line-of-sight or within a pre-determined distance based on the percent probability.

19. The system of claim 18, wherein the wearable medical device comprises one or more magnets that are detected by a plurality of magnetic impedance sensors at specific, known locations in the pre-determined area.

20. The system of claim 18, wherein the wireless communication is selected from at least one of: IEEE 802.11 (WiFi), IEEE 802.15.4, BLUETOOTH protocol, Near Field Communication (NFC), Radio Frequency Identification (RFID), SIGFOX protocol, WiMax (world interoperability for microwave access), Universal Mobile Telecommunications System (UMTS), 3GPP Long Term Evolution (LTE), IMS, High Speed Packet Access (HSPA), Global System for Mobile communication (GSM), 3G, 4G, 5G, 6G and higher, AM, or FM.

21. The system of claim 18, wherein the wearable medical device is powered or passive.

22. The system of claim 18, wherein the processor displays or transfers proximity information to staff in pre-determined area via Zigbee, Bluetooth, WiMax (WiMAX Forum Protocol), Wi-Fi (Wi-Fi Alliance Protocol), GSM (Global System for Mobile Communication), PCS (Personal Communications Services protocol), D-AMPS (Digital-Advanced Mobile Phone Service Protocol), 6LoWPAN (IPv6 Over Low Power Wireless Personal Area Networks Protocol), ANT (ANT network protocol), ANT+, Z-Wave, DASH7 (DASH7 Alliance Protocol), EnOcean, INSTEON, NeuRF ON, Senceive, WirelessHART (Wireless Highway Addressable Remote Transducer Protocol), Contiki, TinyOS (Tiny OS Alliance Protocol), GPRS (General Packet Radio Service), TCP/IP (Transmission Control Protocol and Internet Protocol), CoAP (Constrained Application Protocol), MQTT (Message Queuing Telemetry Transport), TR-50 (Engineering Committee TR-50 Protocol, OMA LW M2M (Open Mobile Alliance LightWeight machine-to-machine Protocol), and ETSIM2M (European Telecommunication Standards Institute machine-to-machine Protocol), Bluetooth Low Energy (BLE), minimal energy Bluetooth signal, Infrared Data Association (IrDA) protocols, and standards related to any of the foregoing.

23. The system of claim 18, wherein the wearable medical device is powered and further comprises at least one of a power source, a display, an input/output device, or a memory.

24. The system of claim 18, wherein the position of the patient or patients is augmented with at least one of: a barometer, a pressure sensor, a vibration sensor, an optical sensor, an infrared sensor, a motion sensor, a magnetometer, or a magnetic sensor.

25. The system of claim 18, wherein the processor is connected to the database via wired or wireless communications, and wherein the database is in the cloud.

26. The system of claim 18, wherein the wearable device transmit signals to the processor for data transmission, transmits a beacon, a broadcast signal, or a packet to the processor for determining the location of the patient.

27. The system of claim 18, wherein the wearable device is worn on a limb or other part of the patient, an accessory worn by the patient, or on a garment worn by the patient.

28. The system of claim 18, further comprising a code segment that anticipates, based on a direction of travel of one or more of the patients with dementia in the pre-determined area, when two patients are likely to come in contact, and alerting staff of the possible contact.

29. The system of claim 18, further comprising a code segment that uses trend analysis to predict at least one of: (1) a long term motion and habit behavior of the one or more patients; (2) trend of a person in the close proximity to another person; (3) determine and track the travel path and location of a specific person; (4) predict where the tracked person may be travelling to a location; or (5) combining trend analysis results to predict a long term motion and habit behavior.

30. The system of claim 29, wherein the code segment for (1) and (5) uses a linear algebraic analysis, wherein the code segment for (3) uses a graph analysis, or wherein the code segment for (2) and (3) uses a Monte-Carlo-based probabilistic prediction.

31. The system of claim 18, further comprising a database that stores the frequency and extend of an altercation two or more patients.

32. The system of claim 18, further comprising a visitor, staff, or family member wearable device to track the position of the visitor, staff, or family member.

33. A wearable device for preventing negative interaction between patients with dementia in a pre-determined area comprising:
a wearable medical device equipped to communicate with the plurality of wireless communication devices actively or passively, wherein the wearable medical device comprises a processor comprising a non-transitory computer readable medium comprising instructions stored thereon for;
communicating with a patient database that comprises specific information for all patients with the wearable medical device, the database further comprising specific information about which patients have dementia, wherein the database includes which patients are likely to have a negative interaction with a dementia patient, and a percent probability that the dementia patient will have a negative interaction with another patient;
using a processor in communication with the patient database and the plurality of wireless communication devices, wherein the processor calculates a position in the pre-determined area for each patient, and
wherein the processor displays an alert when a movement of two patients that are in the patient database having known negative interaction are approaching each other within a line-of-sight or within a pre-determined distance based on the percent probability.
